SEQUENCE LISTING (4 sequences)

SEQ ID NO: 1 - DNA, 20 bp, synthetic construct
  source 1..20: mol_type = other DNA
tgtgcgtgtgtagtgtgtct

SEQ ID NO: 2 - DNA, 20 bp, synthetic construct
  source 1..20: mol_type = other DNA
  modified_base 1: mod_base = OTHER; note = 2'-O-(2-methoxyethyl)-guanosine-3'-monothiophosphat e
  modified_base 2..3: mod_base = OTHER; note = 2'-O-(2-methoxyethyl)-5-methyl-cytidine-3'-monothio phosphate
  modified_base 4: mod_base = OTHER; note = 2'-O-(2-methoxyethyl)-5-methyl-uridine-3'-monothiop hosphate
  modified_base 5: mod_base = OTHER; note = 2'-O-(2-methoxyethyl)-5-methyl-cytidine-3'-monothio phosphate
  modified_base 6: mod_base = OTHER; note = 2'-deoxy-adenosine-3'-monothiophosphate
  modified_base 7: mod_base = OTHER; note = 2'-deoxy-guanosine-3'-monothiophosphate
  modified_base 8: mod_base = OTHER; note = thymidine-3'-monothiophosphate
  modified_base 9: mod_base = OTHER; note = 2'-deoxy-5-methyl-cytidine-3'-monothiophosphate
  modified_base 10: mod_base = OTHER; note = thymidine-3'-monothiophosphate
  modified_base 11: mod_base = OTHER; note = 2'-deoxy-guanosine-3'-monothiophosphate
  modified_base 12: mod_base = OTHER; note = 2'-deoxy-5-methyl-cytidine-3'-monothiophosphate
  modified_base 13..14: mod_base = OTHER; note = thymidine-3'-monothiophosphate
  modified_base 15: mod_base = OTHER; note = 2'-deoxy-5-methyl-cytidine-3'-monothiophosphate
  modified_base 16: mod_base = OTHER; note = 2'-O-(2-methoxyethyl)-guanosine-3'-monothiophosphat e
  modified_base 17: mod_base = OTHER; note = 2'-O-(2-methoxyethyl)-5-methyl-cytidine-3'-monothio phosphate
  modified_base 18: mod_base = OTHER; note = 2'-O-(2-methoxyethyl)-adenosine-3'-monothiophosphat e
  modified_base 19..20: mod_base = OTHER; note = 2'-O-(2-methoxyethyl)-5-methyl-cytidine-3'-monothio phosphate
gcctcagtctgcttcgcacc

SEQ ID NO: 3 - DNA, 21 bp, synthetic construct
  source 1..21: mol_type = other DNA
  modified_base 2: mod_base = um
  modified_base 5..6: mod_base = cm
  modified_base 12: mod_base = um
  modified_base 14..15: mod_base = um
  modified_base 16..17: mod_base = cm
  modified_base 19: mod_base = um
  misc_feature 1..19: note = RNA
  misc_feature 20..21: note = DNA
gtaaccaagagtattccattt

SEQ ID NO: 4 - DNA, 21 bp, synthetic construct
  source 1..21: mol_type = other DNA
  modified_base 7: mod_base = um
  modified_base 17: mod_base = um
  misc_feature 1..19: note = RNA
  misc_feature 20..21: note = DNA
atggaatactcttggttactt

(19) United States
(12) Patent Application Publication
(10) Pub. No.: US 2026/0015657 A1
(43) Pub. Date: Jan. 15, 2026

(54) Nucleic Acid Analytical Method and MALDI Matrix Used for Same

(71) Applicant: SHIMADZU CORPORATION, Kyoto-shi (JP)

(72) Inventor: Yuko FUKUYAMA, Kyoto-shi (JP)

(21) Appl. No.: 18/870,546

(22) PCT Filed: May 31, 2023
(86) PCT No.: PCT/JP2023/020362

§ 371 Date: Nov. 29, 2024

(30) Foreign Application Priority Data
May 31, 2022 (JP) 2022-089238

(51) Int. Cl. C12Q 1/6872

(52) U.S. Cl. CPC C12Q 1/6872

(57) ABSTRACT

Mass spectrometry is performed on a nucleic acid contained in a sample with a matrix-assisted laser desorption/ionization mass spectrometer using a mixed matrix containing 3-hydroxypicolinic acid and 2,4-dihydroxyacetophenone or a mixed matrix containing 3-hydroxypicolinic acid and 2,4,6-trihydroxyacetophenone monohydrate. This makes it possible to detect [M+H]⁺ or [M−H]⁻ of the nucleic acid and fragment ions generated by dissociation of the ions with high sensitivity.

### INCORPORATION BY REFERENCE STATEMENT

This application contains a sequence listing (filename=SHM-1086_replacement_sequence_listing.xml; size=18,811 bytes; date of creation=Aug. 27, 2025) which is herein incorporated by reference in its entirety.

### TECHNICAL FIELD

The present invention relates to a nucleic acid analytical method using mass spectrometry and a MALDI matrix used for the method.

### BACKGROUND ART

Nucleic acids are biopolymers in which nucleotides composed of a base, a sugar, and a phosphoric acid are linked by phosphodiester bonds, and are classified depending on the difference in sugar into deoxyribonucleic acid (DNA) having a 2-deoxy-D-ribose and ribonucleic acid (RNA) having a ribose. Among them, a nucleic acid in which several to several tens of nucleotides are polymerized is also called an oligonucleotide, and can be chemically synthesized. Therefore, in recent years, research to apply oligonucleotides as oligonucleotide therapeutics has been actively conducted, and the importance of analyzing nucleic acids has increased.

A matrix-assisted laser desorption/ionization (MALDI) method is generally known as a soft ionization method capable of ionizing poorly volatile compounds without appreciably decomposing them. A mass spectrometer (MALDI-MS) using this method is widely used to acquire molecular weight information of biopolymers such as peptides, proteins, and sugar chains. However, in the analysis of the nucleic acid using MALDI-MS, the nucleic acid is easily decomposed at the time of ionization. Particularly, the nucleic acid is more easily decomposed as the molecular weight of the nucleic acid is larger, so that a protonated molecule [M+H]⁺ or a deprotonated molecule [M−H]⁻ (M is a molecule, and H is a hydrogen atom) is less likely to be generated. In addition, an alkali metal ion adduct of a nucleic acid is easily generated besides [M+H]⁺ or [M−H]⁻. As a result, there is a problem in that the peak intensity or the S/N ratio of [M+H]⁺ or [M−H]⁻ decreases, and the analytical sensitivity decreases. Therefore, nucleic acid analytical methods capable of detecting these ions with high sensitivity have been studied in various manners.

For example, Non Patent Literature 1 describes a molecular weight analysis of a nucleic acid by a time-of-flight (TOF) mass spectrometer having a MALDI ion source (MALDI-TOFMS). In the molecular weight analysis, by adding ammonium citrate to a matrix, the peak intensity of an alkali metal ion adduct decreased, and [M+H]⁺ of the nucleic acid having a molecular weight of about 6000 could be detected at S/N higher than that before addition of ammonium citrate. Non Patent Literature 2 describes a molecular weight analysis of a nucleic acid by MALDI-TOFMS. In the molecular weight analysis, [M−H]⁻ of RNA having a molecular weight of about 6800 was detected by adding ammonium citrate to 2,4-dihydroxyacetophenone (2,4-DHAP) serving as a matrix.

Non Patent Literature 3 describes a molecular weight analysis of a nucleic acid by MALDI-TOFMS. In the molecular weight analysis, [M−H]⁻ of DNA (21 bases in length) having a molecular weight of about 6000 was detected with high sensitivity by using a mixed matrix containing anthranilic acid (AA) and nicotinic acid (NA).

On the other hand, in a structural analysis of a nucleic acid, [M+H]⁺ or [M−H]⁻ generated from nucleic acid by ionization is intentionally dissociated by an appropriate method using MALDI-MS, and various fragment ions generated by the dissociation are subjected to mass spectrometry to determine their identification, and the molecular structure of the original nucleic acid is estimated. As one of the ion dissociation methods at that time, in-source decay (ISD) is known. The in-source decay is a technique of dissociating ions in an ion source simultaneously with or immediately after ionization. As described above, since the MALDI method is a soft ionization method, it is difficult for ions to dissociate. However, it is known that increasing the ionization energy, for example, by increasing the laser intensity, or using a special matrix promotes dissociation of ions at the time of ionization.

For example, Non Patent Literature 4 describes a structural analysis of a nucleic acid having a relatively short chain of 10 bases in length. In the structural analysis, fragment ions by ISD (ISD fragment ions) centered on w-series ions were detected particularly in a negative ion mode by using 6-thioguanine (TG) as a matrix. Non Patent Literature 5 describes a structural analysis of a relatively short-chain nucleic acid having 10 bases in length. In the structural analysis, a plurality of ISD fragment ions such as a-B, d, y, and w series ions were detected particularly in a negative ion mode by using 1,5-diaminonaphthalene (1,5-DAN) as a matrix. Non Patent Literature 2 describes a structural analysis of a nucleic acid having a relatively long-chain nucleic acid having 20 bases in length. In the structural analysis, ISD fragment ions centered on w-series ions were detected particularly in a negative ion mode by using 2,4-dihydroxyacetophenone (2,4-DHAP) as a matrix.

Non Patent Literature 6 describes a structural analysis of a synthetic oligonucleotide using MALDI-TOFMS. In the structural analysis, a large number of ISD fragment ions of y-series ions were detected by using a mixed matrix containing anthranilic acid (AA) and 3-hydroxypicolinic acid (3-HPA). Non Patent Literature 6 further describes a structural analysis of synthetic oligonucleotide by MALDI-MS (IR-MALDI) using an IR (infrared) laser. In the structural analysis, ISD fragment ions of y-series ions were detected using a mixed matrix containing 2,5-dihydroxybenzoic acid (DHB) or anthranilic acid (AA) and nicotinic acid (NA).

### CITATION LIST

### Non Patent Literature

Non Patent Literature 1: Yoshihiro Wada, “Basis of genome analysis by MS—with SNPs as the center—”, Journal of the Mass Spectrometry Society of Japan, 2003, Vol. 51, No. 2, pp. 368-373

Non Patent Literature 2: H. Shimizu, and six others, “Application of high-resolution ESI and MALDI mass spectrometry to metabolite profiling of small interfering RNA duplex”, Journal of Mass Spectrometry, (USA), 2012, 47, pp. 1015-1022

Non Patent Literature 3: Li-Kang Zhang, and one other, “Matrix-assisted laser desorption/ionization mass spectrometry methods for oligodeoxynucleotides: Improvements in matrix, detection limits, quantification, and sequencing”, Journal of the American Society for Mass Spectrometry, (USA), 2000, 11, pp. 854-865

Non Patent Literature 4: Satoshi Kimura and one other, “Effect of oligonucleotide structural difference on matrix-assisted laser desorption/ionization in-source decay in comparison with collision-induced dissociation fragmentation”, (USA), Rapid Communications in Mass Spectrometry, 2020, 34, e8819

Non Patent Literature 5: Nathan A. Hagan, and five others, “Enhanced In-Source Fragmentation in MALDI-TOF-MS of Oligonucleotides Using 1,5-Diaminonapthalene”, Journal of the American Society for Mass Spectrometry, (USA), 2012, 23, pp. 773-777

Non Patent Literature 6: M. Sebela, “Analysis and sequencing of nucleic acids by matrix-assisted laser desorption/ionisation mass spectrometry”, Spectroscopy Europe, 2016, 28, 5, pp.11-15

Non Patent Literature 7: Scott A. McLuckey and two others, “Tandem mass spectrometry of small, multiply-charged oligonucleotides”, Journal of the American Society for Mass Spectrometry, (USA), 1992, 3, pp. 60-70

### SUMMARY OF INVENTION

### Technical Problem

Regarding the molecular weight analysis of a nucleic acid, in the methods described in Non Patent Literatures 1 and 2, [M+H]⁺ and [M−H]⁻ of relatively high mass nucleic acids are detected using matrix additives, but their sensitivity is still insufficient, and there is room for improvement. In addition, in the matrix and the analytical method described in Non Patent Literature 3, [M−H]⁻ of relatively high mass nucleic acids is detected with high sensitivity, but versatility is limited, and further examination is desired. In addition, in general, matrices that have been reported to be able to be used for nucleic acid analysis are limited. Therefore, a new matrix capable of more effectively detecting [M+H]⁺ or [M−H]⁻ of nucleic acid with high sensitivity is desired.

Regarding the structural analysis of a nucleic acid, in Non Patent Literatures 2 and 4 to 6, by using a matrix having a characteristic of easily generating ISD, ISD fragment ions are detected, and the sequence of nucleic acids is analyzed. However, there is a problem in that the detection sensitivity is low, the number of ion species to be detected or a high-sensitivity peak is limited to a part of the sequence (or mass region), or analysis becomes complicated by detecting a plurality of ion species. Moreover, a decrease in sensitivity causes a decrease in resolution, which makes analysis difficult.

It is considered that one of the reasons why the sensitivity of ISD fragment ions is low is that [M+H]⁺ or [M−H]⁻, which are precursor ions generating ISD fragment ions, are not detected with sufficiently high sensitivity. In addition, regarding the ISD fragment ions, when the number of peaks sufficient for analysis is detected with a resolution sufficient for analysis in addition to the sensitivity in a state where the ion species are unified as much as possible, the structural analysis of a nucleic acid becomes easier. However, matrices reported to be able to be used for structural analysis of nucleic acids (that is, matrices for ISD) are limited, and new matrices are desired.

The present invention has been made in view of the above-described problems, and an object of the present invention is to provide an analytical method capable of detecting [M+H]⁺ or [M−H]⁻ of a nucleic acid and fragment ions generated by dissociation of these ions with high sensitivity, and easily and reliably performing molecular weight analysis and structural analysis of the nucleic acid, and a matrix used for the same.

### Solution to Problem

A nucleic acid analytical method according to the present invention, which has been made to solve the above problems, includes
performing mass spectrometry on a nucleic acid contained in a sample with a matrix-assisted laser desorption/ionization mass spectrometer using a mixed matrix containing 3-hydroxypicolinic acid and 2,4-dihydroxyacetophenone or a mixed matrix containing 3-hydroxypicolinic acid and 2,4,6-trihydroxyacetophenone monohydrate.

A matrix for matrix-assisted laser desorption/ionization mass spectrometry of a nucleic acid according to the present invention, which has been made to solve the above problems, contains 3-hydroxypicolinic acid and 2,4-dihydroxyacetophenone, or contains 3-hydroxypicolinic acid and 2,4,6-trihydroxyacetophenone monohydrate.

### Advantageous Effects of Invention

With the nucleic acid analytical method according to the present invention and the matrix used for the method, a sufficient amount of [M+H]⁺ or [M−H]⁻ of the nucleic acid is generated, and they can be detected with high sensitivity. In addition, with the analytical method according to the present invention and the matrix used for the method, a sufficient amount of [M+H]⁺ or [M−H]⁻ of the nucleic acid is generated, and under the modified analytical conditions optimized for structural analysis, a sufficient amount of fragment ions generated by dissociation from [M+H]⁺ or [M−H]⁻ is also generated, so that fragment ions can be detected with high sensitivity. As a result, the molecular weight analysis and the structural analysis of the nucleic acid can be easily and reliably performed.

### BRIEF DESCRIPTION OF DRAWINGS

FIG. 1 A schematic configuration diagram showing an example of a mass spectrometer (MALDI-ITMS) used for a nucleic acid analytical method according to an embodiment of the present invention.

FIG. 2 A flowchart showing a structural analysis procedure in the nucleic acid analytical method according to the embodiment of the present invention.

FIG. 3 Diagrams showing a mass spectrum of a standard nucleic acid A when various matrices are used in First Reference Example.

FIG. 4 Diagrams showing a mass spectrum of a standard nucleic acid A when various matrices (mixed matrices) are used in First Reference Example.

FIG. 5 A diagram showing a mass spectrum (ISD spectrum) of fragment ions derived from a standard nucleic acid A when 2,4-DHAP is used as a matrix in Second Reference Example.

FIG. 6 Diagrams showing a mass spectrum of mipomersen when various matrices are used and a table showing a detection status of [M+H]⁺ in First Example.

FIG. 7 A diagram showing a mass spectrum of mipomersen when mixed matrix 3-HPA+2,4-DHAP mixed at each matrix ratio is used and a table showing a detection status of [M+H]⁺ in Second Example.

FIG. 8 Diagrams showing a mass spectrum of mipomersen when each matrix is used in Third Example (1).

FIG. 9 Diagrams showing a mass spectrum of mipomersen when each matrix is used in Third Example (2).

FIG. 10 A diagram showing a mass spectrum of mipomersen when a mixed matrix 3-HPA+2,4-DHAP containing ACD at various concentrations is used, and a table showing a detection status of [M+H]⁺ in Fourth Example.

FIG. 11 A diagram showing a mass spectrum (ISD spectrum) of fragment ions derived from mipomersen when a mixed matrix 3-HPA+2,4-DHAP, which is obtained by ISD measurement under the mass-to-charge ratio range condition of mode 3 (m/z 2000 to 18000) is used in Fifth Example.

FIG. 12 A diagram showing a mass spectrum (ISD spectrum) of fragment ions derived from mipomersen when a mixed matrix 3-HPA+2,4-DHAP, which is obtained by ISD measurement under the mass-to-charge ratio range condition of mode 2 (m/z 650 to 5000) is used in Fifth Example.

FIG. 13 ISD spectra of mipomersen when a mixed matrix 3-HPA+2,4-DHAP, which is obtained by ISD measurement under the mass-to-charge ratio range condition of mode 3 (m/z 2000 to 18000), is used in FIGS. 13(a) and 2,4-DHAP is used in FIG. 13(b), in Fifth Example.

FIG. 14 Diagrams showing a mass spectrum of patisiran (1:1 mixture of sense and antisense) when various matrices are used and a table showing a detection status of [M+H]⁺ in Sixth Example.

FIG. 15 Diagrams showing a mass spectrum of patisiran (1:1 mixture of sense and antisense) when various matrices are used and a table showing a detection status of [M+H]⁺ in Sixth Example.

FIG. 16 Diagrams showing a mass spectrum of patisiran (1:1 mixture of sense and antisense) when various matrices are used and a table showing a detection status of [M+H]⁺ in Sixth Example.

FIG. 17 Diagrams showing a mass spectrum of patisiran (1:1 mixture of sense and antisense) when various matrix solutions containing ACD of 40 mM, 70 mM or 100 mM and ACN of 50% or 70% are used and a table showing a detection status of [M+H]⁺ in Seventh Example.

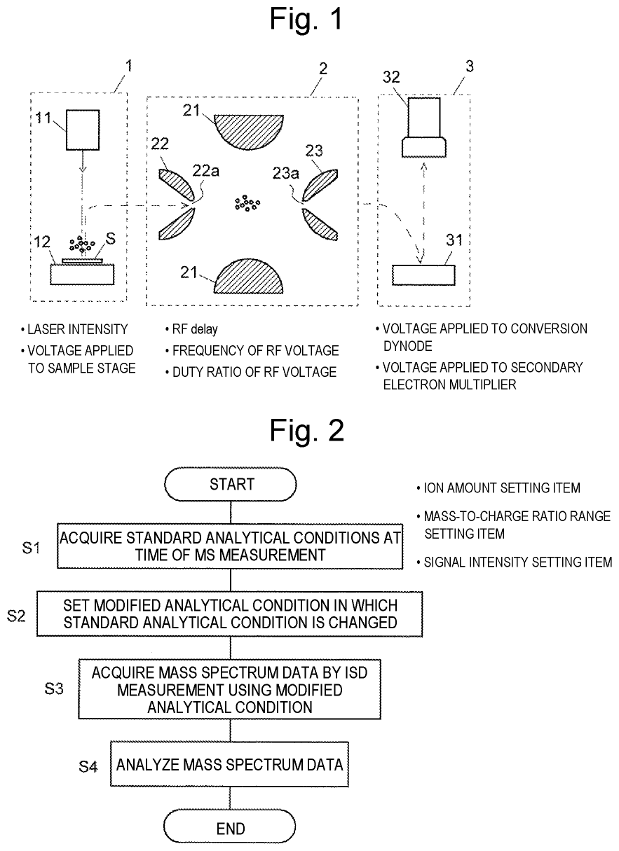

### DESCRIPTION OF EMBODIMENTS

Hereinafter, an embodiment of a nucleic acid analytical method according to the present invention will be described.

### Nucleic Acid

A nucleic acid to be analyzed in the present embodiment includes nucleic acid-related substances such as a modified nucleic acid, a nucleic acid derivative, and an oligonucleotide therapeutics in addition to a nucleic acid. Hereinafter, the nucleic acid and the nucleic acid-related substances are collectively referred to simply as a nucleic acid. The degree of polymerization (bases in length) of the nucleic acid is not particularly limited, but an oligonucleotide in which about several to several tens of nucleotides are polymerized is preferable. In the analytical method according to the present embodiment, since the analytical sensitivity of a nucleic acid having a large molecular weight is particularly improved, a nucleic acid having a molecular weight of 3000 or higher, particularly a nucleic acid having a molecular weight of 6000 or higher is more preferable. In addition, the nucleic acid may be a natural product obtained from an organism or a processed product of the natural product, or may be a chemically synthesized artificial synthetic nucleic acid.

### Mixed Matrix

The mixed matrix used in the present embodiment is a mixed matrix containing 3-hydroxypicolinic acid (3-HPA) and 2,4-dihydroxyacetophenone (2,4-DHAP) (hereinafter, referred to as 3-HPA+2,4-DHAP) or a mixed matrix containing 3-hydroxypicolinic acid (3-HPA) and 2,4,6-trihydroxyacetophenone monohydrate (THAP) (hereinafter, referred to as 3-HPA+THAP). When the nucleic acid to be analyzed is DNA, a mixed matrix containing 3-hydroxypicolinic acid (3-HPA) and 2,4-dihydroxyacetophenone (2,4-DHAP) is preferable, and when the nucleic acid to be analyzed is RNA, a mixed matrix containing 3-hydroxypicolinic acid (3-HPA) and 2,4,6-trihydroxyacetophenone monohydrate (THAP) is preferable. The reason why the suitable mixed matrix is different depending on whether the analysis target is DNA or RNA as described above is considered to be that the affinity with the matrix is also different since the type of sugar contained is different between DNA and RNA.

The mixing ratio of the mixed matrix is not particularly limited, but when the mixed matrix contains 3-HPA and 2,4-DHAP, from the viewpoint that [M+H]⁺ or [M−H]⁻ of the nucleic acid and fragment ions generated by dissociation of the preceding ions (precursor ions) can be detected with high sensitivity, preferably 3-HPA: 2,4-DHAP is 10:1 to 1:5, more preferably 7:1 to 1:5, and particularly preferably 5:1 to 1:1. When the mixed matrix contains 3-HPA and THAP, preferably 3-HPA: THAP is 1:1 to 1:5, and more preferably 1:1 to 1:3 from the same viewpoint.

The mixed matrix may further contain, as a matrix additive, ammonium citrate dibasic; ACD). There are several types of ammonium salts of citric acid depending on the number of ammonium ions bonded to the citrate ion, and a salt in which two ammonium ions are bonded to one citrate ion is suitably used in the present embodiment. The concentration of the matrix additive in the mixed matrix is preferably 10 to 100 mM, more preferably 30 to 90 mM, and still more preferably 40 to 85 mM from the viewpoint of being able to detect [M+H]⁺ or [M−H]⁻ of the nucleic acid and fragment ions generated by dissociation of the preceding ions (precursor ions) with high sensitivity. In the present specification, a numerical range from a lower limit value to an upper limit value is indicated as “(lower limit value) to (upper limit value)” using the word “to”, and the numerical range indicated in this manner includes the lower limit value itself and the upper limit value itself.

As a method for preparing a sample for analysis, a mixed solution in which a sample containing nucleic acid and a mixed matrix are mixed is dried on a sample plate of a mass spectrometer. The mixed solution may be prepared in advance, and the mixed solution may be dripped onto a sample plate and dried, or the mixed solution may be prepared on a sample plate and dried as it is.

### Mass Spectrometer

The mass spectrometer used in the present embodiment is a mass spectrometer (MALDI-MS) having an ion source performing a MALDI method. Examples of the MALDI-MS include time-of-flight MALDI-TOFMS and ion trap type MALDI-ITMS. The ion trap type MALDI-MS herein is a mass spectrometer having an ion trap for capturing ions. Specifically, the ion trap type mass spectrometer herein includes a mass spectrometer configured to discharge, in ascending order of mass-to-charge ratio (m/z), ions captured in the ion trap using a mass separation function of the ion trap itself, and detect the ions by a detector disposed outside the ion trap. In addition, the ion trap type mass spectrometer includes a mass spectrometer configured to separate ions simultaneously discharged from the ion trap in accordance with the mass-to-charge ratio by a mass separation unit disposed outside the ion trap, such as a time-of-flight mass separation unit, for example, and detect the ions by a detector disposed similarly outside the ion trap.

### Structural Analysis of Nucleic Acid

The analytical method according to the present embodiment may include: a data acquisition step of performing mass spectrometry on a sample containing a nucleic acid using the above-described mixed matrix to acquire mass spectrum data of a plurality of fragment ions generated by dissociation of [M+H]⁺ or [M−H]⁻ of the nucleic acid, and a data analysis step of extracting peaks of a plurality of fragment ions derived from the nucleic acid from the mass spectrum data acquired in the data acquisition step and determining a structure of the nucleic acid based on mass information of the peaks. This makes it possible to perform structural analysis of the nucleic acid. Hereinafter, a method for performing structural analysis will be described assuming that the mass spectrometer is an ion trap type MALDI-ITMS.

### Ion Trap Type Mass Spectrometer

The ion trap type mass spectrometer includes: an ion source 1 configured to ionize a sample containing an object to be analyzed; an ion trap 2 (ion capturing unit in the present invention) configured to temporarily capture ions having a predetermined mass-to-charge ratio among ions generated by the ion source 1 and separate the captured ions in accordance with the mass-to-charge ratio (m/z) by the action of a radio-frequency electric field; and a detection unit 3 configured to detect the separated ions.

The ion source 1 is an ion source using the MALDI method, and includes a laser irradiation unit 11 configured to irradiate a sample with laser light and a sample stage 12 on which a sample plate S having the sample on it is placed. The ion trap 2 is a quadrupole ion trap including a ring electrode 21 of an annular shape and a pair of end cap electrodes 22 and 23 disposed to face each other across the ring electrode 21. An ion incidence hole 22a is formed in the inlet-side end cap electrode 22, and an ion emission hole 23a is formed in the outlet-side end cap electrode 23. The detector 3 includes a conversion dynode 31 configured to convert ions into electrons and a detector (secondary electron multiplier) 32 configured to multiply and detect electrons arriving from the conversion dynode 31.

A corresponding predetermined voltage is applied to each of the ring electrode 21 and the end cap electrodes 22 and 23 of the ion trap 2. Due to the radio-frequency electric field thus formed, ions can be captured in the internal space surrounded by the ring electrode 21 and the end cap electrodes 22 and 23, and ions can be discharged from the internal space through the ion emission hole 23a.

The range of the mass-to-charge ratio of ions preferentially captured in the ion trap is controlled by changing the time (hereinafter referred to as delay time) from when the sample is irradiated with laser light by the ion source 1 to when a capturing voltage for capturing ions is applied to the ring electrode 21 of the ion trap 2. When the delay time is lengthened, ions on the higher mass side can be captured more reliably than other ions, and when the delay time is shortened, ions on the lower mass side can be captured more reliably than other ions.

The predetermined voltage applied to the ring electrode 21 and the end cap electrodes 22 and 23 may be a sinusoidal radio-frequency voltage or a rectangular wave voltage generated by switching two different voltages at high speed. In the digital ion trap using the electric field generated by the rectangular wave voltage, the range of the mass-to-charge ratio of ions that can be captured is controlled by changing the frequency while maintaining the amplitude (voltage value) of the rectangular wave voltage constant, or by changing the duty ratio that is the ratio of the switching interval of the rectangular wave voltage.

Next, a procedure for performing nucleic acid structural analysis will be described with reference to the flowchart of FIG. 2.

### Structural Analysis Method

Here, an example in which MALDI-ITMS having a digital ion trap is used as the mass spectrometer will be described.

### Step 1: Acquisition of Standard Analytical Conditions at the Time of MS Measurement

First, standard analytical conditions are acquired when a measurement (hereinafter, referred to as MS measurement) for detecting a protonated molecule [M+H]⁺ or a deprotonated molecule [M−H]⁻ of a nucleic acid is performed. The MS measurement corresponds to a first measurement in the present invention. The MS measurement is a measurement without ion dissociation (or even if ion dissociation occurs, it is very slight) since a measurement condition for detecting [M+H]⁺ or [M−H]⁻ with as high sensitivity and resolution as possible is used. The mass spectrometer includes various setting items that can be appropriately set according to the type of the object to be analyzed, the purpose of analysis, and the like. In the structural analysis method of the present embodiment, standard analytical conditions for detecting [M+H]⁺ or [M−H]⁻ of the object to be analyzed by MS measurement are acquired for an ion amount setting item regarding the amount of ions generated by the ion source 1, a mass-to-charge ratio range setting item regarding the mass-to-charge ratio of ions captured in the ion trap 2, and a signal intensity setting item regarding the signal intensity of ions in the detection unit 3.

The standard analytical condition is a typical condition among conditions under which [M+H]⁺ or [M−H]⁻ of the nucleic acid to be analyzed is detectable. As a method for acquiring standard analytical conditions, for example, when [M+H]⁺ or [M−H]⁻ of the nucleic acid as an object to be analyzed is detected as a result of MS measurement performed using default values of various setting items set in advance in the mass spectrometer, the default values may be used as standard analytical conditions. In that case, reading the default values from a storage unit or the like in which the default values are stored in advance corresponds to acquiring standard analytical conditions. Alternatively, MS measurement may be performed by changing setting values of various setting items with respect to default values so that [M+H]⁺ or [M−H]⁻ of the nucleic acid is detected with higher sensitivity and higher resolution, and values (a threshold and the like) at which the ion is detected may be obtained.

The ion amount setting item includes the laser intensity (laser power) emitted by the laser irradiation unit 11, the mass-to-charge ratio range setting item includes a RF delay value corresponding to the delay time, and the signal intensity setting item includes the voltage applied to the conversion dynode 31, the voltage applied to the detector (secondary electron multiplier) 32, and the like.

### Step 2: Setting of Modified Analytical Conditions in Which Standard Analytical Condition is Changed

Next, among the standard analytical conditions acquired in step 1, a modified analytical condition obtained by changing at least one of the ion amount setting item, the mass-to-charge ratio range setting item, and the signal intensity setting item is set. That is, there may be a case where only one of the ion amount setting item, the mass-to-charge ratio range setting item, and the signal intensity setting item is changed, and the remaining setting items are not changed. At this time, the ion amount setting item is changed so that the amount of ions generated in the ion source 1 increases, the mass-to-charge ratio range setting item is changed so that ions on the low mass side are preferentially captured in the ion trap 2, and the signal intensity setting item is changed so that the signal intensity of ions increases in the detection unit 3, as compared with the case where MS measurement is performed under standard analytical conditions.

Specifically, when the intensity of the laser light, which is the ion amount setting item, is changed, it is preferable to set the intensity higher than the standard analytical condition, for example, it is more preferable to set the intensity higher by about 1 to 40% than the value of the standard analytical condition, and it is still more preferable to set the intensity higher by about 1 to 30% than the value of the standard analytical condition. When the RF delay value, which is a mass-to-charge ratio range setting item, is changed, it is preferable to set the RF delay value to be lower than the standard analytical condition, for example, it is more preferable to set the RF delay value to be lower by 5 to 30% than the value of the standard analytical condition, and it is still more preferable to set the RF delay value to be lower by 10 to 20% (so that the delay time is 1 to 3 μs). When the voltage applied to the conversion dynode 31, which is the signal intensity setting item, is changed, it is preferable to set the voltage higher than the standard analytical condition, for example, it is preferable to set the voltage higher by about 5 to 30%, and it is more preferable to set the voltage higher by about 10 to 30%. When the voltage applied to the detector (secondary electron multiplier) 32, which is also the signal intensity setting item, is changed, it is preferable to set the voltage higher than the standard analytical condition, for example, it is preferable to set the voltage higher by about 5 to 50%, and it is more preferable to set the voltage higher by about 10 to 50%.

### Step 3: Acquisition of Mass Spectrum Data by ISD Measurement Using Modified Analytical Conditions

Measurement for detecting fragment ions (decomposition products) derived from nucleic acids is performed using the above-described modified analytical conditions. In the present embodiment, a generation mechanism of fragment ions derived from an object to be analyzed (nucleic acid) has not yet been proved. Therefore, in the present specification, dissociation simultaneously with or immediately after ionization in the ion source of the MALDI ion trap type mass spectrometer and overall dissociation of ions generated in the subsequent apparatus are referred to as in-source decay (ISD), and measurement for detecting fragment ions derived from the nucleic acid dissociated by ISD is referred to as ISD measurement. The ISD measurement corresponds to a second measurement in the present invention. Mass spectrum data of a plurality of fragment ions generated by dissociation of [M+H]⁺ or [M−H]⁻ (precursor ions) of a nucleic acid can be acquired by performing ISD measurement using the above-described modified analytical conditions. In particular, fragment ions in the low mass region away from m/z of [M+H]⁺ or [M−H]⁻ can be detected with high sensitivity.

In step 3, the ISD measurement may be further performed using conditions in which the predetermined mass-to-charge ratio range is set such that the maximum value of the predetermined mass-to-charge ratio of the ions captured in the ion trap 2 is smaller than the value of the mass-to-charge ratio of [M+H]⁺ or [M−H]⁻ of the nucleic acid that is object to be analyzed. At this time, it is preferable that the maximum value of the predetermined mass-to-charge ratio of the ions captured in the ion trap 2 is set to be smaller than the value of the mass-to-charge ratio of [M+H]⁺ or [M−H]⁻ of the object to be analyzed by, for example, about 0.5 to 40%, and more preferably by about 0.5 to 20%.

As a method of changing the predetermined mass-to-charge ratio range, for example, there is a method of switching a measurement mode incorporated in a device for an object to be analyzed having a molecular weight of about 6000 from a measurement mode in which a target mass region is m/z 2000 to 18000 to a measurement mode in which the target mass region is m/z 650 to 5000, thereby switching to a measurement mode not including a molecular weight of 6000 as an object. The target mass region of the measurement mode is determined according to the frequency of the radio-frequency voltage applied to the ion trap 2. Specifically, since the amount of ions captured in the ion trap is limited, the mass range to be measured is determined mainly by adjusting the frequency of the radio-frequency voltage and setting a low mass cut-off value (LMCO). When the frequency of the radio-frequency voltage is increased, the LMCO is set to be small, and the mass region to be measured is set on the low mass side. On the other hand, when the frequency of the radio-frequency voltage is decreased, the LMCO is set to be large, and the mass region to be measured is set on the high mass side. That is, the range of the predetermined mass-to-charge ratio is changed by changing the frequency of the radio-frequency voltage applied to the ion trap 2. In addition, as an example method of changing the predetermined mass-to-charge ratio range, there is a method of excluding ions of m/z 5500 or higher by changing a value of a setting item of a duty ratio, which is a ratio of a switching interval of a rectangular wave voltage incorporated in a device, for an object to be analyzed having a molecular weight of about 6000, to, for example, specifically, a value of 52:48 from a standard value of 50:50. By doing so, mass spectrum data in which fragment ions having a mass-to-charge ratio close to m/z of precursor ions are excluded is obtained. As a result, the detection sensitivity of fragment ions in a low mass region away from m/z of precursor ions is improved.

In step 3, the ISD measurement may be performed both under the condition in which the predetermined mass-to-charge ratio range is set as described above and under the condition in which the predetermined mass-to-charge ratio range is not used to acquire the mass spectrum data obtained in each measurement.

In addition to using the condition in which the predetermined mass-to-charge ratio range is set such that the maximum value of the predetermined mass-to-charge ratio of the ions captured in the ion trap 2 is smaller than the value of the mass-to-charge ratio of [M+H]⁺ or [M−H]⁻ of the nucleic acid that is the object to be analyzed, a standard analytical condition may be further acquired in step 2 for the voltage applied to the sample stage 12 of the ion source 1, and the ISD measurement may be performed using the condition in which the voltage applied to the sample stage 12 is changed to a larger value than the analytical condition. More preferably, the voltage applied to the sample stage is set to be 4 to 8 times higher than, and still more preferably set to be 4 to 5 times higher than standard analytical conditions.

### Step 4: Mass Spectrum Data Analysis

Peaks corresponding to various fragment ions are extracted from the mass spectrum data obtained in step 3, identification of various fragment ions is determined based on the mass information indicated by the peaks, and the results are combined to determine at least a part of the structure of the original nucleic acid. The determination of the structure includes sequence analysis and specifying the type of chemical modification or the site subjected to the chemical modification by the sequence analysis. Database search or De Novo Sequencing may be used to determine the structure.

In step 3, when mass spectrum data is acquired both under the condition where the predetermined mass-to-charge ratio range is set and under the condition where the predetermined mass-to-charge ratio range is not set, analysis may be performed by combining identification results of various fragment ions obtained under both the conditions. By doing so, it is possible to more reliably perform structural analysis of the nucleic acid that is an object to be analyzed.

Hereinafter, an analytical method according to the present invention will be described by way of example, but this is merely an example, and the present invention is not limited to this.

### EXAMPLES

### First Reference Example

First, a matrix used as a comparative object of the present invention was selected.

### 1. Preparation of Sample Solution

As a sample solution, a 10 pmol/μL aqueous solution of standard nucleic acid A(5′-TGTGCGTGTGTAGTGTGTCT-3′: SEQ ID NO. 1, 20 bases in length, MW 6201.1, synthesis request product) was prepared.

### 2. Preparation of Matrix Solution

As a matrix solution, a 40 mg/mL 50% acetonitrile (ACN) aqueous solution of each of 3-hydroxypicolinic acid (3-HPA), 2,4-dihydroxyacetophenone (2,4-DHAP), 2,4,6-trihydroxyacetophenone monohydrate (THAP), and 1,5-diaminonaphthalene (1,5-DAN) containing ammonium citrate dibasic (ACD) at a concentration of 70 mM as a matrix additive was prepared.

As a mixed matrix solution, a 0.02 mmol/50 μL 50% ACN aqueous solution of anthranilic acid (AA) containing ACD at a concentration of 70 mM and a 0.01 mmol/50 μL 50% ACN aqueous solution of nicotinic acid (NA) containing ACD at a concentration of 70 mM were prepared, and they were mixed at 1:1 (v/v) (2:1 (mol/mol)) to prepare a mixed matrix (AA+NA) solution. A 50 mg/mL 50% ACN aqueous solution of anthranilic acid (AA) and 3-hydroxypicolinic acid (3-HPA) containing ACD at a concentration of 70 mM was made and mixed at 1:1 (v/v) to make a mixed matrix (AA+3-HPA) solution.

### 3. Preparation of Sample for Analysis

The sample solution prepared in 1. and the matrix solution or mixed matrix solution prepared in 2. were mixed at a ratio of 1:1 (v/v), and 1 μL of the obtained mixed solution was dripped onto a sample plate (SUS plate) and dried.

### 4. Mass Spectrometry

For the mass spectrometry, a MALDI digital ion trap type mass spectrometer (MALDI-DITMS, manufactured by Shimadzu Corporation, trade name: MALDImini-1) was used. The sample plate on which the sample for analysis prepared in 3. was placed was inserted into MALDI-DITMS, and MS measurement was performed using raster-function in positive mode.

### Results

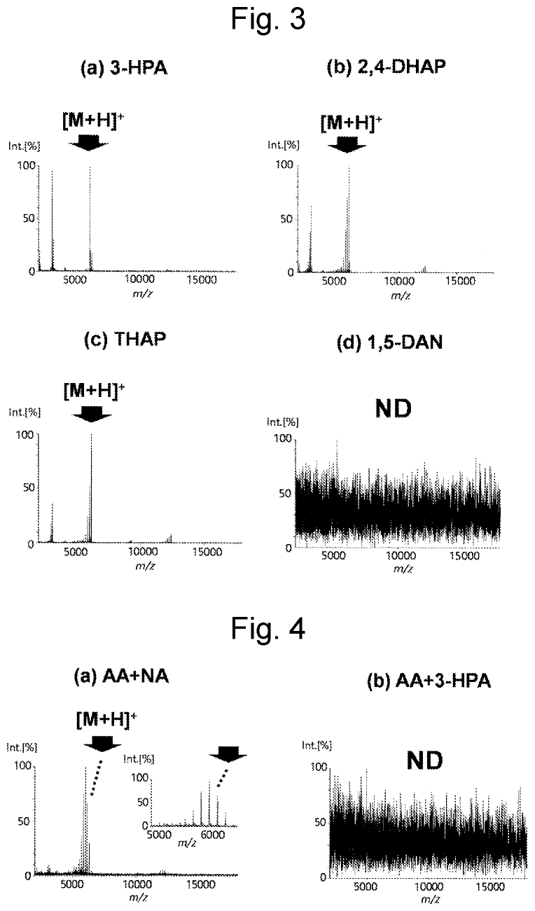

FIG. 3 shows mass spectra of standard nucleic acids A when 3-HPA, 2,4-DHAP, THAP, and 1,5-DAN in FIG. 3(a), (b), (c), and (d) are used as matrices, respectively. The arrow in FIG. 3 indicates the detection status of [M+H]⁺. When the peak of [M+H]⁺ is not detected, the detection status is indicated as ND (not detected).

From FIG. 3, when 3-HPA, 2,4-DHAP, and THAP in FIG. 3(a), (b), and (c) were used as matrices, respectively, a peak of [M+H]⁺ was detected, but when 1,5-DAN was used in FIG. 3(d), a peak of [M+H]⁺ was not detected. As a matrix, 1,5-DAN has been reported to be used for analyzing a relatively short-chain nucleic acid having about 10 bases in length (10 mer), and it is considered that the standard nucleic acid A having 18 bases in length (18 mer) was not able to be ionized by 1,5-DAN.

FIG. 4 shows mass spectra of standard nucleic acids A when AA+NA and AA+3-HPA in FIG. 4(a) and (b) are used as mixed matrices, respectively. An enlarged diagram of the mass spectrum is also shown in FIG. 4(a). From FIG. 4, when AA+NA was used in FIG. 4(a), the peak of [M+H]⁺ was detected, and many base elimination ions and alkali metal ion adducts were detected together with the peak of [M+H]⁺. The detection sensitivity of [M+H]⁺ was lower than that in the case of using 3-HPA in FIG. 3(a), 2,4-DHAP in FIG. 3(b), and THAP in FIG. 3(c) were used. When AA+3-HPA in FIG. 4(b) was used, the peak of [M+H]⁺ of the standard nucleic acid A was not detected. In FIG. 4(a), AA+NA is a mixed matrix reported as a matrix for high sensitivity analysis of high mass nucleic acids, but at least under the present conditions, the sensitivity was lower than 3-HPA, 2,4-DHAP, and THAP. In addition, in FIG. 4(b), AA+3-HPA is reported as a matrix for ISD of nucleic acids, but ionization itself of nucleic acid molecules was difficult at least under the current conditions.

From the above results, 3-HPA, 2,4-DHAP, and THAP were selected as matrices to be used for comparison.

### Second Reference Example

Next, ISD measurement of nucleic acid was performed using 2,4-DHAP as a matrix.

### 1. Preparation of Sample Solution

As a sample solution, a 10 pmol/μL aqueous solution of standard nucleic acid A was prepared.

### 2. Preparation of Matrix Solution

As a matrix solution, a 40 mg/mL 50% ACN aqueous solution of 2,4-dihydroxyacetophenone (2,4-DHAP) containing ACD at a concentration of 70 mM as a matrix additive was prepared.

### 3. Preparation of Sample for Analysis

The sample solution prepared in 1. and the matrix solution prepared in 2. were mixed at a ratio of 1:1 (v/v), and 1 μL of the obtained mixed solution was dripped onto a sample plate (SUS plate) and dried.

### 4. Mass Spectrometry

For the mass spectrometry, a MALDI digital ion trap type mass spectrometer (MALDI-DITMS, manufactured by Shimadzu Corporation, trade name: MALDImini-1) was used. The sample plate on which the sample for analysis prepared in 3. was placed was inserted into MALDI-DITMS, and ISD measurement was performed using a raster-function in positive mode. Analytical conditions when performing ISD measurement are as follows. The measurement mode indicates the range of the mass-to-charge ratio of the ion to be measured, specifically, specifies the range of the mass-to-charge ratio of the ion captured in the ion trap, and the range is indicated in parentheses.
Measurement mode: mode 3 (m/z 2000 to 18000)
Detector voltage (DV-1): 2000
Conversion voltage (DV-2): 8000
RF delay value (RF): 17
Laser power (LP): 75
Sample stage voltage (SV): 5
Duty ratio: 50:50

### 5. Data Analysis

The mass spectrum data (ISD spectrum) of the fragment ions obtained by the ISD measurement in 4. was subjected to determination of identification of main peaks.

### Results

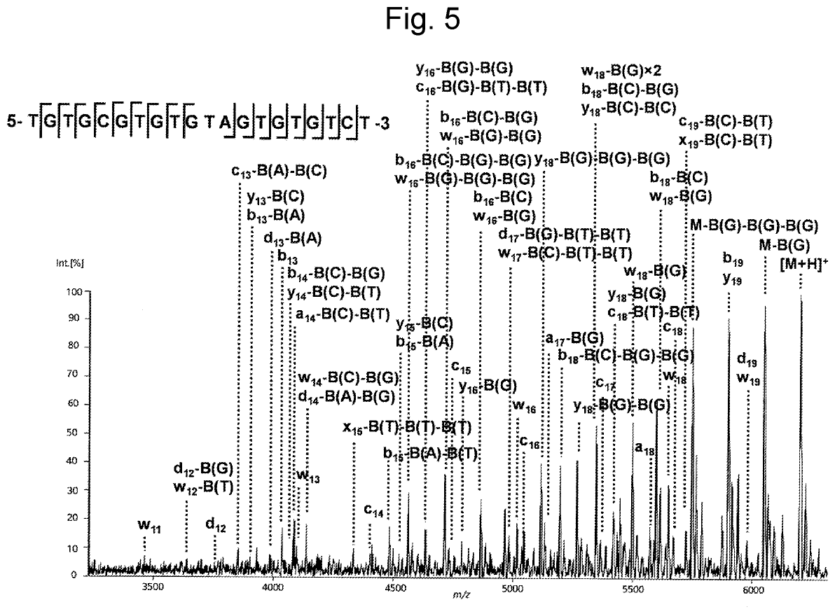

FIG. 5 shows an ISD spectrum of a standard nucleic acid A obtained by ISD measurement using 2,4-DHAP as a matrix. In FIG. 5, each peak in the mass spectrum is given the name of the fragment ion species of the corresponding oligonucleotide (general name proposed in Non Patent Literature 7). The name is a name representing each ion species in a fragment ion sequence according to a nucleic acid dissociation pattern naming rule. In this naming rule, fragment ions including the 5′-end are denoted as aₙ, bₙ, cₙ, and dₙ, and fragment ions at the 3′-end in the opposite direction are denoted as xₘ, yₘ, zₘ, and wₘ. Note that the suffixes n and m indicate the number of constitutional units (the number of bases by definition) from the corresponding end to the dissociation site. In addition, B in the identifier represents a base of a nucleic acid, and for example, b₁₃-B(A) in FIG. 5 indicates an ion in which adenine (A) of the base has been eliminated from a b₁₃-ion. The same applies to the following drawings.

When 2,4-DHAP was used as a matrix, many peaks in which a base had been eliminated were observed in the obtained ISD spectrum, and a plurality of fragment ion species were mixed, which was complicated. In the MALDI-ITMS used in this measurement, it is known that fragmentation easily occurs due to the device structural characteristics as compared with the conventional MALDI-TOFMS. Since it is not easy to analyze the base sequence from such a mass spectrum, in order to speed up the analysis, a simpler ISD spectrum in which base elimination is suppressed and ion species are unified to some extent is desired.

Hereinafter, MS measurement of mipomersen known as one of oligonucleotide therapeutics was performed by the analytical method according to the present invention.

### First Example

### 1. Preparation of Sample Solution

As sample solutions, a 20 pmol/μL aqueous solution of mipomersen (single-stranded DNA, 5′-MG-MC-MC-MU-MC-dA-dG-dT-dC-dT-dG-dC-dT-dT-dC-MG-MC-MA-MC-MC-3′ (M represents 2′-O-(2-methoxyethyl)nucleoside, and d represents 2′-deoxynucleoside. The carbon in position 5 of each of cytosine and uracil is substituted with a methyl group, and phosphodiester bonds between all nucleotides are substituted with phosphorothioate bonds.): SEQ ID NO. 2, 20 bases in length, MW 7177, sample after desalting purification of synthetic nucleic acid for research and development) was prepared.

### 2. Preparation of Matrix Solution

As a matrix solution, a 40 mg/mL 50% ACN aqueous solution (3-HPA solution) of 3-hydroxypicolinic acid (3-HPA) containing ACD at a concentration of 40 mM as a matrix additive was prepared.

A 40 mg/mL 50% ACN aqueous solution (THAP solution) of 2,4,6-trihydroxyacetophenone monohydrate (THAP) containing ACD at a concentration of 40 mM as a matrix additive was prepared.

A 40 mg/mL 50% ACN aqueous solution (2,4-DHAP solution) of 2,4-dihydroxyacetophenone (2,4-DHAP) containing ACD at a concentration of 70 mM was prepared.

As a mixed matrix solution, a mixed matrix (3-HPA+2,4-DHAP) solution was prepared by mixing the 3-HPA solution and the 2,4-DHAP solution at a ratio of 1:1 (v/v). Similarly, a mixed matrix (3-HPA+THAP) solution was prepared by mixing the 3-HPA solution and the THAP solution at a ratio of 1:1 (v/v).

### 3. Preparation of Sample for Analysis

The sample solution prepared in 1. and the matrix solution and the mixed matrix solution prepared in 2. were mixed at a ratio of 1:1 (v/v), and 1 μL of the obtained mixed solution was dripped onto a sample plate (SUS plate) and dried.

### 4. Mass Spectrometry

For the mass spectrometry, a MALDI digital ion trap type mass spectrometer (MALDI-DITMS, manufactured by Shimadzu Corporation, trade name: MALDImini-1) was used. The sample plate on which the sample for analysis prepared in 3. was placed was inserted into MALDI-DITMS, and MS measurement was performed using raster-function in positive mode.

### Results

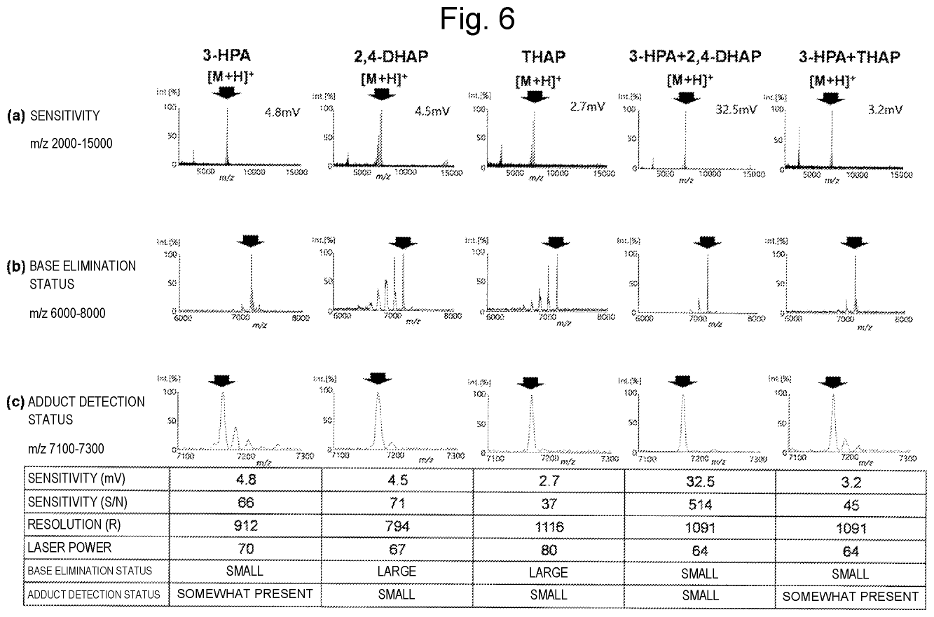

FIG. 6 shows mass spectra (3-HPA, 2,4-DHAP, THAP, 3-HPA+2,4-DHAP, 3-HPA+THAP in order from the left) of mipomersen when 3-HPA, 2,4-DHAP, and THAP are used as matrices and 3-HPA+2,4-DHAP and 3-HPA+THAP are used as mixed matrices. The upper part, FIG. 6(a), is an overall diagram showing a region of m/z 2000 to 15000, the middle part, FIG. 6(b), is an enlarged diagram showing a region of m/z 6000 to 8000, and the lower part, FIG. 6(c), is an enlarged diagram showing a region of m/z 7100 to 7300. The arrow in FIG. 6 indicates the detection status of [M+H]⁺. Further, a table summarizing details (sensitivity (mV, S/N), resolution (R), base elimination status, adduct detection status) of the detection status of [M+H]⁺ and laser power at the time of measurement when each matrix is used is shown below the mass spectrum. The base elimination status was judged from the mass spectrum of FIG. 6(b), and the adduct detection status was judged from the mass spectrum of FIG. 6(c).

From FIG. 6, when the mixed matrix 3-HPA+2,4-DHAP was used, [M+H]⁺ was detected with the highest sensitivity and a sufficient resolution in a state in which base elimination and formation of an alkali metal adduct were suppressed. As a result, it was confirmed that the mixed matrix 3-HPA+2,4-DHAP was particularly effective for MS measurement of nucleic acids (DNA) of 20 bases or more in length.

### Second Example

Mass spectrometry was performed in the same manner as in First Example except that the method for preparing the matrix solution was different, and the mixing ratio of the mixed matrix 3-HPA+2,4-DHAP was examined.

### 2. Preparation of Matrix Solution

A 40 mg/mL 50% ACN aqueous solution (3-HPA solution) of 3-hydroxypicolinic acid (3-HPA) containing ACD at a concentration of 70 mM as a matrix additive was prepared.

A 40 mg/mL 50% ACN aqueous solution (2,4-DHAP solution) of 2,4-dihydroxyacetophenone (2,4-DHAP) containing ACD at a concentration of 70 mM as a matrix additive was prepared.

The 3-HPA solution and the 2,4-DHAP solution that have been prepared were mixed at a desired mixing ratio (v/v) to prepare a mixed matrix 3-HPA+2,4-DHAP (mixing ratio (1:0), (10:1), (7:1), (5:1), (3:1), (2:1), (1:1), (1:2), (1:3), (1:5), (1:7), (1:10), (0:1)) solution. At this time, the mixing ratio (1:0) indicates that 3-HPA is used alone, and the mixing ratio (0:1) indicates that 2,4-DHAP is used alone.

### Results

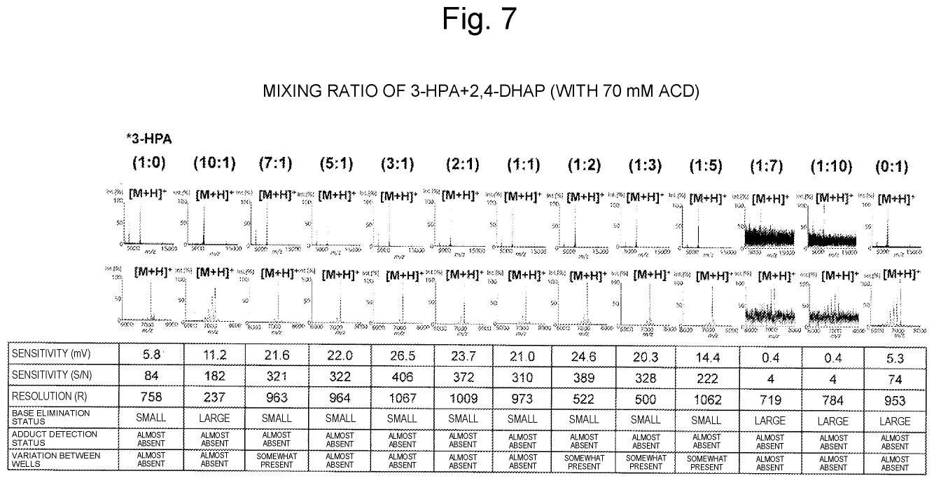

FIG. 7 shows a table summarizing the mass spectra of mipomersen when 3-HPA+2,4-DHAP (mixing ratios (1:0), (10:1), (7:1), (5:1), (3:1), (2:1), (1:1), (1:2), (1:3), (1:5), (1:7), (1:10), and (0:1) in order from the left is used as a mixed matrix, and details of the detection status (sensitivity (mV, S/N), resolution (R), base elimination status, adduct detection status, and variation between wells) of the detection status of [M+H]⁺ when a mixing matrix of each mixing ratio is used.

As compared with the case of using 3-HPA+2,4-DHAP (mixing ratio (1:0), (0:1)), that is, 3-HPA or 2,4-DHAP alone, the detection sensitivity of [M+H]⁺ was improved in the case of using the mixed matrix 3-HPA+2,4-DHAP prepared at a mixing ratio (10:1) to (1:5). In addition, when the mixed matrix 3-HPA+2,4-DHAP prepared at a mixing ratio (7:1) to (1:5) was used, base elimination was suppressed in addition to improvement in detection sensitivity. In consideration of reproducibility such as variation between wells, it was confirmed that when a mixed matrix 3-HPA+2,4-DHAP prepared at a mixing ratio (5:1) to (1:1) was used, [M+H]⁺ was able to be reproducibly detected with high sensitivity and high resolution and in a state in which base elimination and production of an alkali metal ion adduct were suppressed.

### Third Example (1) and Third Example (2)

Mass spectrometry was performed in the same manner as in First Example except that the method for preparing the matrix solution was different, and the effect of isomer of 2,4-DHAP was examined.

### Third Example (1)

### 2. Preparation of Matrix Solution

A 40 mg/mL 50% ACN aqueous solution (3-HPA solution) of 3-hydroxypicolinic acid (3-HPA) containing ACD at a concentration of 40 mM as a matrix additive was prepared.

A 40 mg/mL 50% ACN aqueous solution (2,4-DHAP solution) of 2,4-dihydroxyacetophenone (2,4-DHAP) containing ACD at a concentration of 70 mM as a matrix additive was prepared.

A 40 mg/mL 50% ACN aqueous solution (2,5-DHAP solution) of 2,5-DHAP, which is a regioisomer of 2,4-DHAP, containing ACD at a concentration of 70 mM as a matrix additive was prepared.

The 3-HPA solution and the 2,4-DHAP solution that have been prepared, and the 3-HPA solution and the 2,5-DHAP solution that have been prepared were each mixed at a ratio of 1:1 (v/v) to prepare mixed matrix (3-HPA+2,4-DHAP (1:1) and 3-HPA+2,5-DHAP (1:1) solutions.

### Results

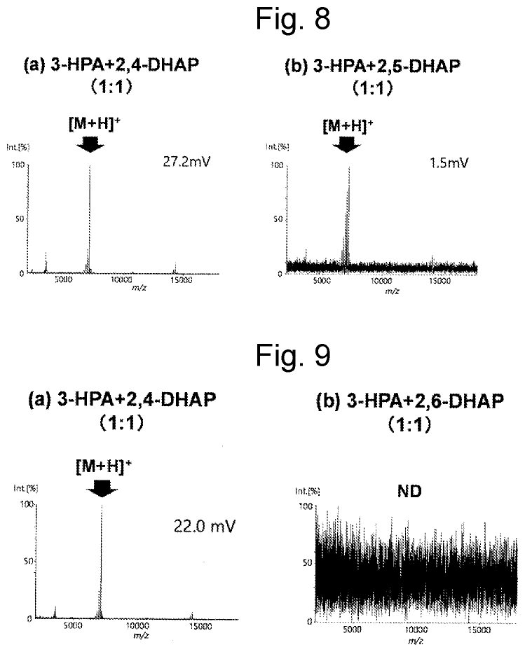

FIG. 8 shows mass spectra of mipomersen when mixed matrix 3-HPA+2,4-DHAP (1:1) and 3-HPA+2,5-DHAP (1:1) are used. The arrow in FIG. 8 indicates the detection status of [M+H]⁺. The numerical values in FIG. 8 indicate the peak intensity (mV) of [M+H]⁺. The sensitivity of [M+H]⁺ was lower when 3-HPA+2,5-DHAP (1:1) was used than when 3-HPA+2,4-DHAP (1:1) was used.

### Third Example (2)

### 2. Preparation of Matrix Solution

A 40 mg/mL 50% ACN aqueous solution (3-HPA solution) of 3-hydroxypicolinic acid (3-HPA) containing ACD at a concentration of 70 mM as a matrix additive was prepared.

A 40 mg/mL 50% ACN aqueous solution (2,4-DHAP solution) of 2,4-dihydroxyacetophenone (2,4-DHAP) containing ACD at a concentration of 70 mM as a matrix additive was prepared.

A 40 mg/mL 50% ACN aqueous solution (2,6-DHAP solution) of 2,6-DHAP, which is a regioisomer of 2,4-DHAP, containing ACD at a concentration of 70 mM as a matrix additive was prepared.

The 3-HPA solution and the 2,4-DHAP solution that have been prepared and the 3-HPA solution and the 2,6-DHAP solution that have been prepared were each mixed at a mixing ratio 1:1 (v/v) to prepare a mixed matrix (3-HPA+2,4-DHAP (1:1) and 3-HPA+2,6-DHAP (1:1)) solutions.

### Results

FIG. 9 shows mass spectra of mipomersen when mixed matrix 3-HPA+2,4-DHAP (1:1) and 3-HPA+2,6-DHAP (1:1) are used. When 3-HPA+2,4-DHAP (1:1) was used, [M+H]⁺ of mipomersen was detected with high sensitivity, but when 3-HPA+2,6-DHAP (1:1) was used, ionization itself of nucleic acid molecules was difficult.

### Fourth Example

Mass spectrometry was performed in the same manner as in First Example except that the method for preparing the matrix solution was different, and concentration of ammonium citrate dibasic (ACD), which is a matrix additive, was examined.

### 2. Preparation of Matrix Solution

A 40 mg/mL 50% ACN aqueous solution (3-HPA solution) of 3-hydroxypicolinic acid (3-HPA) containing ACD at a concentration of each of 40, 70, and 100 mM as a matrix additive was prepared.

A 40 mg/mL 50% ACN aqueous solution (2,4-DHAP solution) of 2,4-dihydroxyacetophenone (2,4-DHAP) containing ACD at a concentration of each of 40, 70, and 100 mM as a matrix additive was prepared.

A mixed matrix (3-HPA+2,4-DHAP (40, 55, 70, 85, and 100 mM ACD)) solution was prepared by mixing the 3-HPA solution and the 2,4-DHAP solution containing ACD at a corresponding concentration at a ratio of 1:1 (v/v). Specifically, a 3-HPA (40 mM ACD) solution and a 2,4-DHAP (40 mM ACD) solution were mixed at 1:1 (v/v) to prepare a mixed matrix (3-HPA+2,4-DHAP) solution containing 40 mM ACD. Similarly, a 3-HPA (40 mM ACD) solution and a 2,4-DHAP (70 mM ACD) solution, a 3-HPA (70 mM ACD) solution and a 2,4-DHAP (70 mM ACD) solution, a 3-HPA (70 mM ACD) solution and a 2,4-DHAP (100 mM ACD) solution, and a 3-HPA (100 mM ACD) solution and a 2,4-DHAP (100 mM ACD) solution were each mixed at 1:1 (v/v) to prepare a mixed matrix (3-HPA+2,4-DHAP) solution containing 55, 70, 85, and 100 mM ACD.

### Results

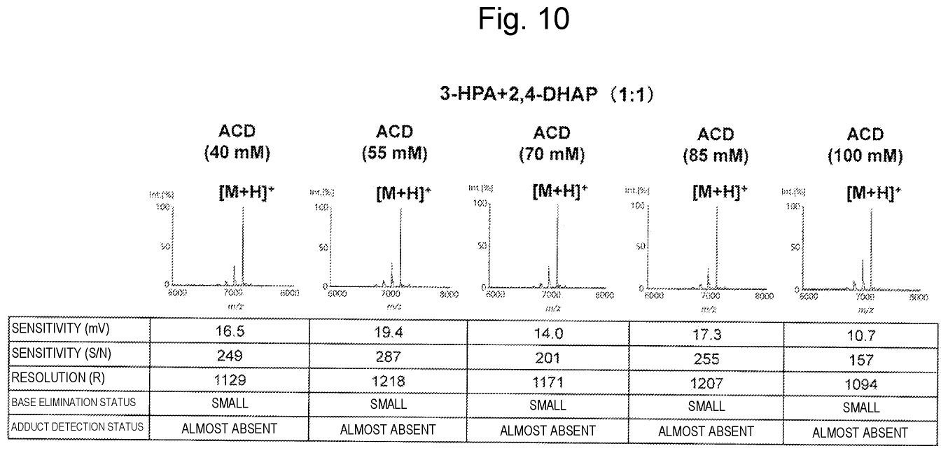

FIG. 10 shows a table summarizing the mass spectrum of mipomersen when using a mixed matrix 3-HPA+2,4-DHAP of each ACD concentration (40, 55, 70, 85, 100 mM) and the detection status (sensitivity (mV, S/N), resolution (R), adduct detection status, base elimination status) of [M+H]⁺ when using each mixed matrix.

Even when a mixed matrix having any ACD concentration was used, [M+H]⁺ was detected with high sensitivity and high resolution and in a state in which base elimination and formation of an alkali metal ion adduct were suppressed. It was confirmed that the detection sensitivity slightly decreased when the ACD concentration was 100 mM, but a similarly favorable mass spectrum was obtained when the ACD concentration was 40 to 85 mM.

### Fifth Example

Next, ISD measurement of mipomersen was performed by the analytical method according to the present invention.

### 1. Preparation of Sample Solution

As a sample solution, a 20 pmol/μL aqueous solution of mipomersen was prepared.

### 2. Preparation of Matrix Solution

A 40 mg/mL 50% ACN aqueous solution (3-HPA solution) of 3-hydroxypicolinic acid (3-HPA) containing ACD at a concentration of 40 mM as a matrix additive was prepared.

A 40 mg/mL 50% ACN aqueous solution (2,4-DHAP solution) of 2,4-dihydroxyacetophenone (2,4-DHAP) containing ACD at a concentration of 70 mM as a matrix additive was prepared.

A mixed matrix (3-HPA+2,4-DHAP (1:1)) solution was prepared by mixing the 3-HPA solution and the 2,4-DHAP solution that have been prepared at a ratio of 1:1 (v/v).

### 3. Preparation of Sample for Analysis

The sample solution prepared in 1. and the mixed matrix solution prepared in 2. were mixed at a ratio of 1:1 (v/v), and 1 μL of the obtained mixed solution was dripped onto a sample plate (SUS plate) and dried.

### 4. Mass Spectrometry

For the mass spectrometry, a MALDI digital ion trap type mass spectrometer (MALDI-DITMS, manufactured by Shimadzu Corporation, trade name: MALDImini-1) was used. The sample plate on which the sample for analysis prepared in section 3. was placed on it was inserted into MALDI-DITMS, and ISD measurement was performed using a raster function in positive mode. Analytical conditions when performing ISD measurement are as follows. The measurement mode indicates the range of the mass-to-charge ratio of the ion to be measured, specifically, specifies the range of the mass-to-charge ratio of the ion captured in the ion trap, and the range is indicated in parentheses. As the laser power, optimum values were correspondingly used.
Measurement mode: mode 3 (m/z 2000 to 18000), mode 2 (m/z 650 to 5000)
Detector voltage (DV-1): 2000
Conversion voltage (DV-2): 8000
RF delay value (RF): 17
Sample stage voltage (SV): 5
Duty ratio: 50:50

### 5. Data Analysis

The mass spectrum data (ISD spectrum) of the fragment ions obtained by the ISD measurement in 4. was subjected to identification of main peaks.

### Results

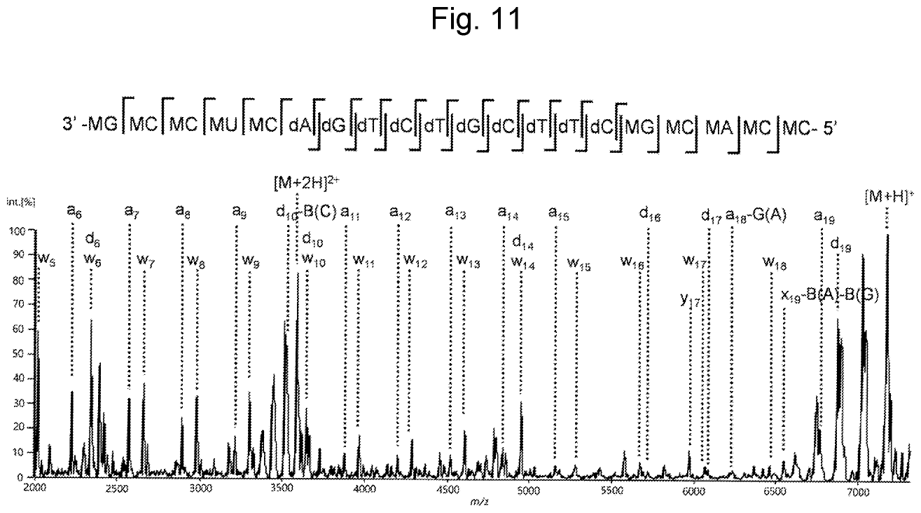

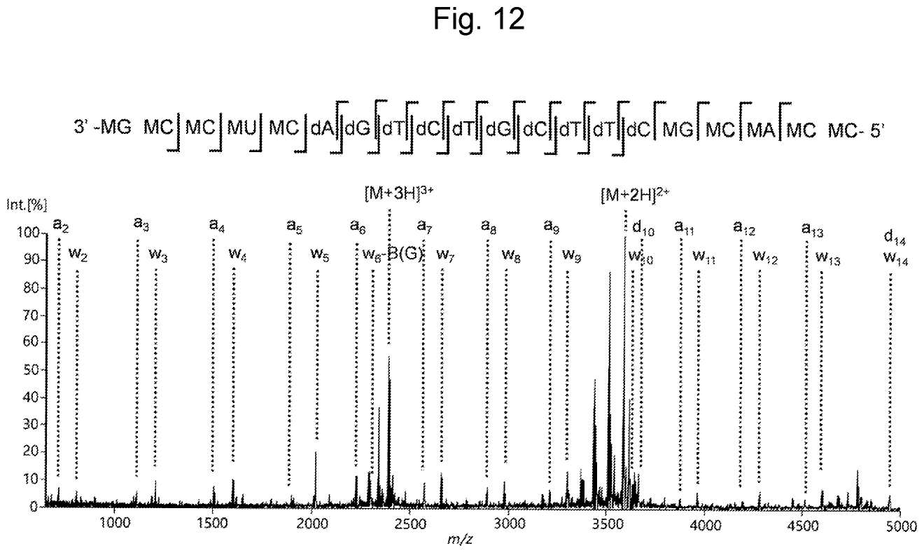

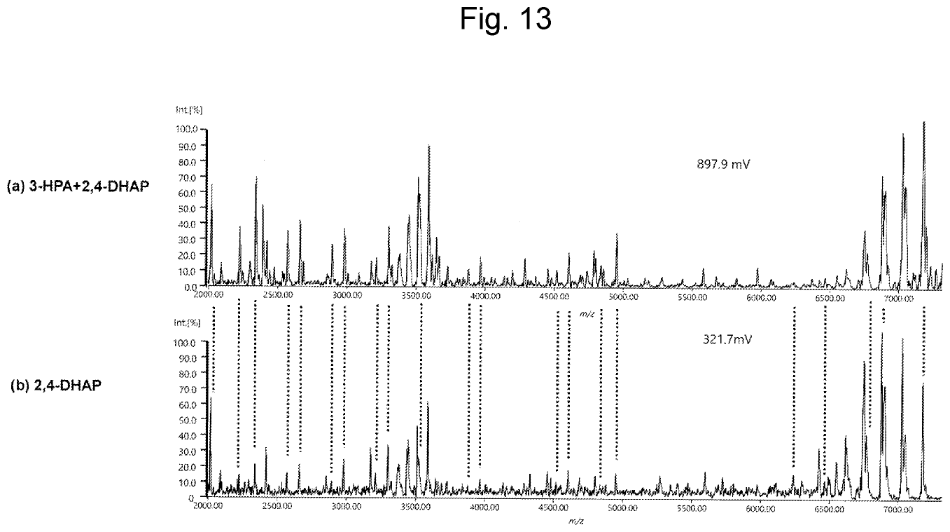

FIG. 11 shows an ISD spectrum and an identification status of main peaks of mipomersen obtained by ISD measurement under the mass-to-charge ratio range condition of mode 3 when a mixed matrix 3-HPA+2,4-DHAP is used. FIG. 12 shows an ISD spectrum and an identification status of main peaks of mipomersen obtained by ISD measurement under the mass-to-charge ratio range condition of mode 2 when a mixed matrix 3-HPA+2,4-DHAP is used. In FIGS. 11 and 12, a simple ISD spectrum in which a large number of ISD fragment ions centered on a/w-ions were preferentially detected was obtained. As a result, the identification of ISD fragment ions can be easily determined, and analysis can be quickly performed. In addition, by combining the analysis results of FIGS. 11 and 12, the entire sequence of mipomersen was able to be more reliably analyzed.

FIG. 13 shows ISD spectra of mipomersen obtained by ISD measurement under the mass-to-charge ratio range condition of mode 3 when a mixed matrix 3-HPA+2,4-DHAP in FIGS. 13(a) and 2,4-DHAP in FIG. 13(b) are used, respectively. The broken line in FIG. 13 indicates that the peak is detected in both the cases of using these matrices and is assigned in FIG. 11. The sensitivity of ISD fragment ions in the case of using the mixed matrix 3-HPA+2,4-DHAP was higher than that in the case of using 2,4-DHAP, which is a conventional matrix for ISD, and a/w-ions were observed more preferentially. From this, it was confirmed that a simple ISD spectrum capable of easily performing structural analysis can be obtained by using the mixed matrix 3-HPA+2,4-DHAP.

The reason why such a result was obtained is considered to be related to the fact that when MS measurement was performed using the mixed matrix 3-HPA+2,4-DHAP as shown in FIG. 6 of First Example, [M+H]⁺ was detected with high sensitivity and high resolution and in a state in which base elimination and production of an alkali metal ion adduct were suppressed. The ISD measurement is a method in which [M+H]⁺ or [M−H]⁻ is generated as a precursor ion, and then fragment ions generated by dissociation of the precursor ion are subjected to mass spectrometry. Since [M+H]⁺ of the nucleic acid was detected with sufficient sensitivity in MS measurement using the mixed matrix 3-HPA+2,4-DHAP, it is considered that precursor ions that are [M+H]⁺ are generated in a sufficient amount even in ISD measurement using the same mixed matrix. As a result, it is considered that when the mixed matrix has a characteristic of easily generating ISD as in this case, fragment ions generated from precursor ions by ISD are also generated in a sufficient amount, which leads to detection of the fragment ions with high sensitivity. In addition, it is considered that having a characteristic of hardly generating an adduct and not generating dissociation of an unstable site such as base elimination during ionization leads to generation of a simpler ISD fragment species.

### Sixth Example

Next, MS measurement of patisiran known as one of the oligonucleotide therapeutics was performed by the analytical method according to the present invention.

### 1. Preparation of Sample Solution

As a sample solution, patisiran (originally double-stranded RNA composed of sense strand and antisense strand, but a mixture of sense and antisense at 1:1 (mol/mol) was used in this example. A 20 pmol/μL aqueous solution of sense 5′-G-Um-A-A-Cm-Cm-A-A-G-A-G-Um-A-Um-Um-Cm-Cm-A-Um-dT-dT-3′ (dT represents thymidine deoxyribonucleotide, Cm represents 2′-O-methylcytidine, and Um represents 2′-O-methyluridine): SEQ ID NO. 3, 21 bases in length (core sequence has 19 bases in length and a DNA overhang (dTdT) of 2 bases at 3′ end)), MW 6764, and antisense 5′-A-U-G-G-A-A-Um-A-C-U-C-U-U-G-G-U-Um-A-C-dT-dT-3′ (dT represents thymidine deoxyribonucleotide, and Um represents 2′-O-methyluridine): SEQ ID NO: 4, 21 bases in length (core sequence has 19 bases in length and a DNA overhang (dTdT) of 2 bases at 3′ end)), MW 6660, sample after desalting purification of a synthetic nucleic acid for research and development) was prepared.

### 2. Preparation of Matrix Solution

A 40 mg/mL 50% ACN aqueous solution (3-HPA-1 solution) of 3-hydroxypicolinic acid (3-HPA) containing ACD at a concentration of 40 mM as a matrix additive was prepared. A 40 mg/mL 50% ACN aqueous solution (3-HPA-2 solution) of 3-HPA containing ACD at a concentration of 70 mM as a matrix additive was prepared.

A 40 mg/mL 50% ACN aqueous solution (2,4-DHAP solution) of 2,4-dihydroxyacetophenone (2,4-DHAP) containing ACD at a concentration of 70 mM as a matrix additive was prepared.

A 40 mg/mL 50% ACN aqueous solution (THAP-1 solution) of 2,4,6-trihydroxyacetophenone monohydrate (THAP) containing ACD at a concentration of 40 mM as a matrix additive was prepared.

A 40 mg/mL 50% ACN aqueous solution (THAP-2 solution) of THAP containing ACD at a concentration of 70 mM as a matrix additive was prepared.

The prepared 3-HPA-1 solution and 2,4-DHAP solution were mixed at mixing ratios (1:1), (3:1), and (1:3) (v/v) to prepare various mixed matrix (3-HPA-1+2,4-DHAP) solutions ((mixing ratio 1:1, 55 mM ACD), (mixing ratio 3:1, 48 mM ACD), and (mixing ratio 1:3, 63 mM ACD)).

The prepared 3-HPA-2 solution and 2,4-DHAP solution were mixed at mixing ratios (1:1), (3:1), and (1:3) (v/v) to prepare various mixed matrix (3-HPA-2+2,4-DHAP) solutions ((mixing ratio 1:1, 70 mM ACD), (mixing ratio 3:1, 70 mM ACD), and (mixing ratio 1:3, 70 mM ACD)).

The prepared 3-HPA-1 solution and THAP-1 solution were mixed at mixing ratios (1:1), (3:1), and (1:3) (v/v) to prepare various mixed matrix (3-HPA-1+THAP-1) solutions ((mixing ratio 1:1, 40 mM ACD), (mixing ratio 3:1, 40 mM ACD), and (mixing ratio 1:3, 40 mM ACD)).

The prepared 3-HPA-2 solution and THAP-2 solution were mixed at mixing ratios (1:1), (3:1), and (1:3) (v/v) to prepare various mixed matrix (3-HPA-2+THAP-2) solutions ((mixing ratio 1:1, 70 mM ACD), (mixing ratio 3:1, 70 mM ACD), and (mixing ratio 1:3, 70 mM ACD)).

### 3. Preparation of Sample for Analysis

The sample solution prepared in 1. and the mixed matrix solution prepared in 2. were mixed at a ratio of 1:1 (v/v), and 1 μL of the obtained mixed solution was dripped onto a sample plate (SUS plate) and dried.

### 4. Mass Spectrometry

For the mass spectrometry, a MALDI digital ion trap type mass spectrometer (MALDI-DITMS, manufactured by Shimadzu Corporation, trade name: MALDImini-1) was used. The sample plate on which the sample for analysis prepared in 3. was placed was inserted into MALDI-DITMS, and MS measurement was performed using raster-function in positive mode.

### Results

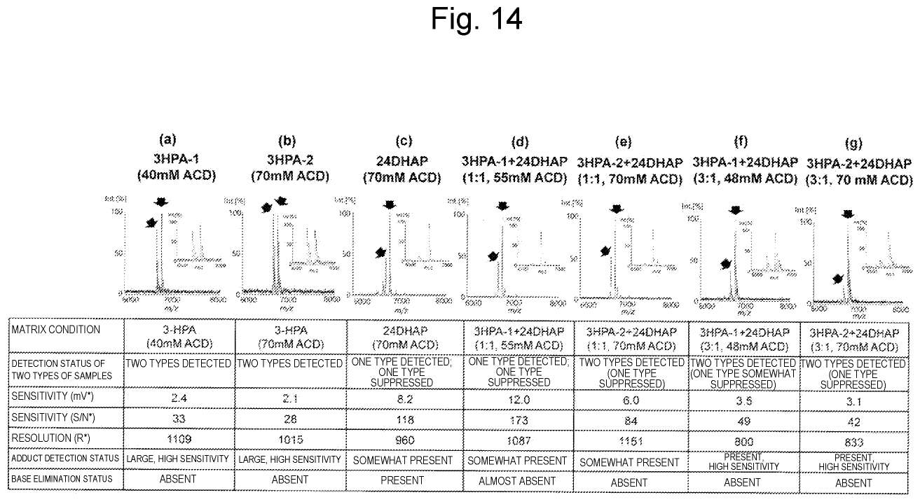

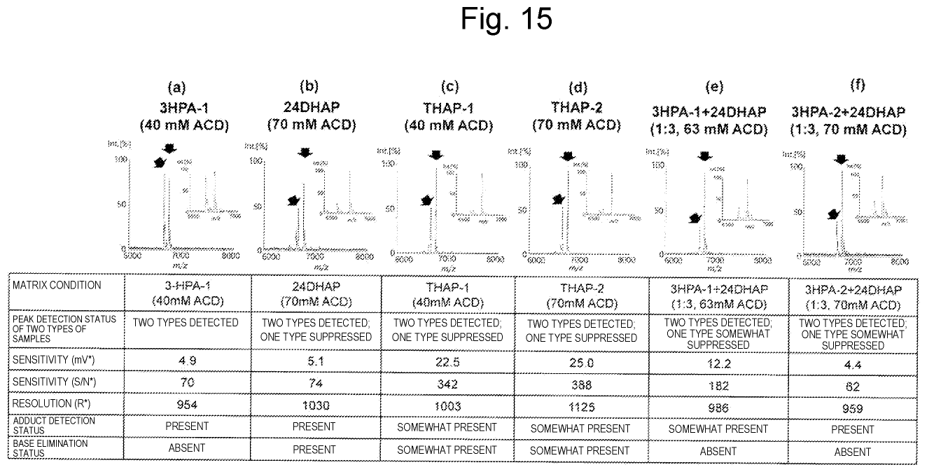

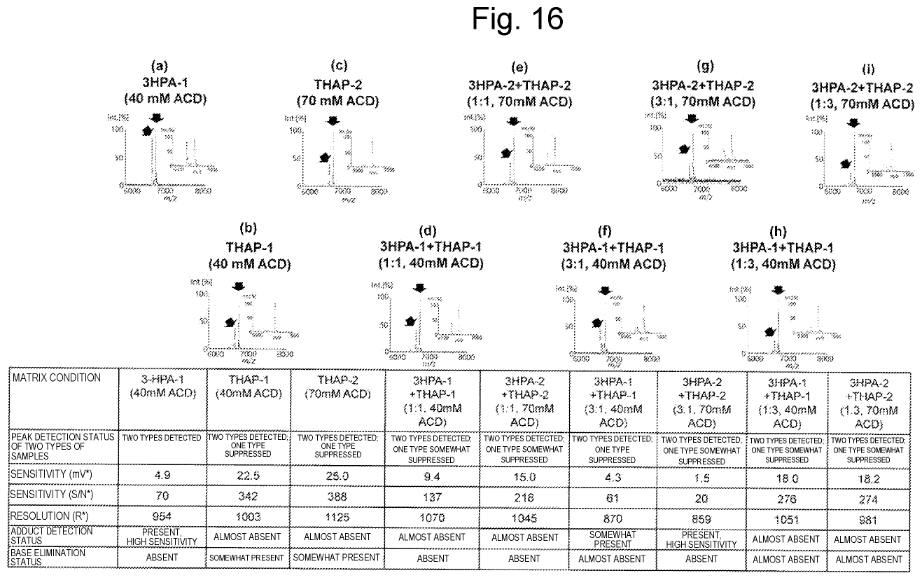

FIGS. 14, 15, and 16 show mass spectra of patisiran in the case of using various matrix solutions and enlarged views of them, and a table summarizing details (detection status, sensitivity (mV, S/N), resolution (R), adduct detection status, and base elimination status of two types (sense and antisense) of samples) of the detection status of [M+H]⁺ of the two types of samples of patisiran. For the sensitivity and resolution, as a representative, numerical values for the [M+H]⁺ peak of sense among the two types of samples are shown. FIGS. 15 and 16 show data acquired on the same day, and FIG. 14 shows data acquired on a different day from the preceding pieces of data.

From FIGS. 14 to 16, it was confirmed that [M+H]⁺ was detected in a mixed matrix in which 3-HPA and THAP were contained at a mixing ratio of 1:1 or 1:3, particularly at a mixing ratio of 1:1, in both types of sense and antisense of patisiran with high sensitivity and high resolution, and in a state in which base elimination and production of an alkali metal ion adduct were suppressed (FIGS. 16(d), (e), (h), and (i)). From this, it was confirmed that the mixed matrix 3-HPA+2,4-THAP was particularly effective for MS measurement of relatively high mass nucleic acids (RNA) of 21 bases in length. As shown in FIG. 6 of First Example, when the nucleic acid sample was mipomersen, which is DNA, a mixed matrix containing 3-HPA and 2,4-DHAP, rather than a mixed matrix containing 3-HPA and THAP, was most suitable for detection of [M+H]⁺. The reason why the suitable types of mixed matrix are different depending on difference in types of nucleic acids as described above is considered to be that the affinity with the matrix is different depending on difference in sugars contained in DNA and RNA.

### Seventh Example

Mass spectrometry was performed in the same manner as in Sixth Example except that the method for preparing the matrix solution was different, and concentration of ammonium citrate dibasic (ACD) and concentration of acetonitrile (ACN) as matrix additives were examined.

### 2. Preparation of Matrix Solution

A 40 mg/mL 50% ACN aqueous solutions (3-HPA-1 solution, 3-HPA-2 solution, and 3-HPA-3 solution in ascending order of concentration of ACD) of 3-hydroxypicolinic acid (3-HPA) containing ACD at concentrations of 40, 70, and 100 mM as a matrix additive were prepared, respectively.

A 40 mg/mL 50% ACN aqueous solutions (THAP-1 solution, THAP-2 solution, and THAP-3 solution in ascending order of concentration of ACD) of 2,4,6-trihydroxyacetophenone (THAP) containing ACD at concentrations of 40, 70, and 100 mM as a matrix additive were prepared, respectively.

A 40 mg/mL 70% ACN aqueous solutions (3-HPA-4 solution, 3-HPA-5 solution, and 3-HPA-6 solution in ascending order of concentration of ACD) of 3-HPA containing ACD at concentrations of 40, 70, and 100 mM as a matrix additive were prepared, respectively.

A 40 mg/mL 70% ACN aqueous solutions (THAP-4 solution, THAP-5 solution, and THAP-6 solution in ascending order of concentration of ACD) of THAP containing ACD at concentrations of 40, 70, and 100 mM as a matrix additive were prepared, respectively.

The prepared 3-HPA-1 to 6 solutions and THAP-1 to 6 solutions were mixed at a mixing ratio of 1:1 (v/v) to prepare a mixed matrix 3-HPA-1+THAP-1 solution (40 mM ACD, 50% ACN/Water), a 3-HPA-2+THAP-2 solution (70 mM ACD, 50% ACN/Water), a 3-HPA-3+THAP-3 solution (100 mM ACD, 50% ACN/Water), a 3-HPA-4+THAP-4 solution (40 mM ACD, 70% ACN/Water), a 3-HPA-5+THAP-5 solution (70 mM ACD, 70% ACN/Water), and a 3-HPA-6+THAP-6 solution (100 mM ACD, 70% ACN/Water), respectively.

### Results

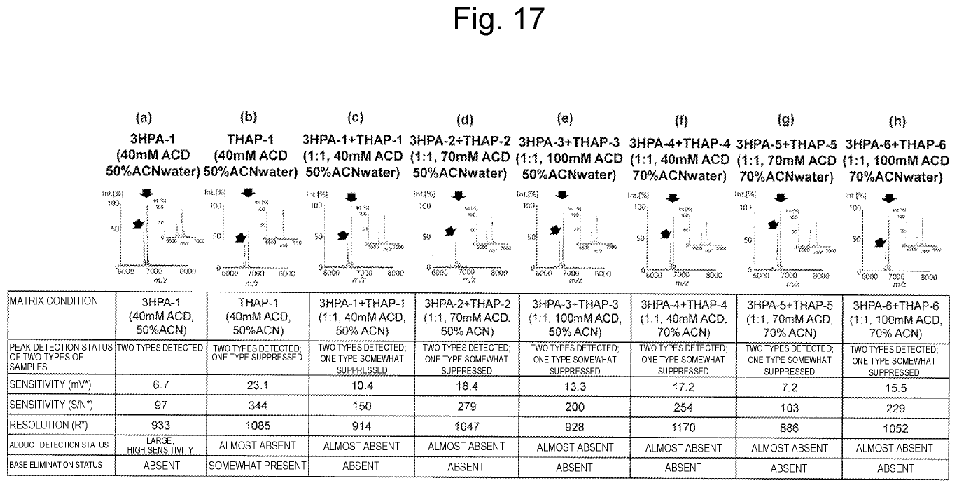

FIG. 17 shows mass spectra of patisiran in the case of using various matrix solutions and enlarged views of them, and a table summarizing details (detection status, sensitivity (mV, S/N), resolution (R), adduct detection status, and base elimination status of two types of samples) of the detection status of [M+H]⁺ of two types (sense and antisense) of samples of patisiran. For the sensitivity and resolution, as a representative, numerical values for the [M+H]⁺ peak of sense among the two types of samples are shown.

From FIG. 17, it was confirmed that when a mixed matrix of 3-HPA and THAP (mixing ratio 1:1) was used, regardless of whether the concentration of ACD was 40 to 100 mM or the concentration of acetonitrile as a solvent is 50 to 70%, [M+H]⁺ was detected with high sensitivity and high resolution in both types of sense and antisense of patisiran and in a state in which base elimination and production of an alkali metal ion adduct were suppressed. Among them, the most favorable [M+H]⁺ peak was obtained when the 3-HPA-2+THAP-2 solution (70 mM ACD, 50% ACN/Water) was used (FIG. 17(d)).

### MODES

A person skilled in the art can understand that the previously described illustrative embodiments are specific examples of the following modes of the present invention.

### Clause 1

A nucleic acid analytical method according to a mode of the present invention includes
performing mass spectrometry on a nucleic acid contained in a sample with a matrix-assisted laser desorption/ionization mass spectrometer using a mixed matrix containing 3-hydroxypicolinic acid and 2,4-dihydroxyacetophenone or a mixed matrix containing 3-hydroxypicolinic acid and 2,4,6-trihydroxyacetophenone monohydrate.

This makes it possible to detect [M+H]⁺ or [M−H]⁻ of the nucleic acid and fragment ions generated by dissociation of the ions with high sensitivity.

### Clause 2

In the nucleic acid analytical method according to clause 1,
when the mixed matrix is a mixed matrix containing 3-hydroxypicolinic acid and 2,4-dihydroxyacetophenone, a mixing ratio of 3-hydroxypicolinic acid and 2,4-dihydroxyacetophenone may be 10:1 to 1:5.

This makes it possible to detect [M+H]⁺ or [M−H]⁻ of the nucleic acid, particularly DNA, and fragment ions generated by dissociation of the ions with higher sensitivity.

### Clause 3

In the nucleic acid analytical method according to clause 1,
when the mixed matrix is a mixed matrix containing 3-hydroxypicolinic acid and 2,4,6-trihydroxyacetophenone monohydrate, a mixing ratio of 3-hydroxypicolinic acid and 2,4,6-trihydroxyacetophenone monohydrate may be 1:1 to 1:3.

This makes it possible to detect [M+H]⁺ or [M−H]⁻ of the nucleic acid, particularly RNA, and fragment ions generated by dissociation of the ions with higher sensitivity.

### Clause 4

In the nucleic acid analytical method according to any one of clauses 1 to 3,
the mixed matrix may further contain ammonium citrate dibasic as a matrix additive.

This makes it possible to detect [M+H]⁺ or [M−H]⁻ of the nucleic acid and fragment ions generated by dissociation of the ions with higher sensitivity.

### Clause 5

The nucleic acid analytical method according to any one of clauses 1 to 4 may include:
a data acquisition step of performing mass spectrometry on a sample containing the nucleic acid using the mixed matrix to acquire mass spectrum data of a plurality of fragment ions generated by dissociation of [M+H]⁺ or [M−H]⁻ of the nucleic acid; and
a data analysis step of extracting peaks of the plurality of fragment ions derived from the nucleic acid from the mass spectrum data and determining a structure of the nucleic acid based on mass information of the peaks.

This makes it possible to perform structural analysis of the nucleic acid.

### Clause 6

In the nucleic acid analytical method according to clause 5,
the matrix-assisted laser desorption/ionization mass spectrometer may be an ion trap type matrix-assisted laser desorption/ionization mass spectrometer including: an ion source performing a matrix-assisted laser desorption/ionization method; an ion capturing unit configured to separate and capture ions having a predetermined mass-to-charge ratio from ions generated by the ion source; and a detection unit configured to detect ions captured by the ion capturing unit,
the nucleic acid analytical method may include an analytical condition acquisition step of acquiring standard analytical conditions of an ion amount setting item regarding an amount of ions generated by the ion source, a mass-to-charge ratio range setting item regarding a mass-to-charge ratio range of ions captured by the ion capturing unit, and a signal intensity setting item regarding signal intensity of ions in the detection unit when a first measurement for detecting [M+H]⁺ or [M−H]⁻ of the nucleic acid contained in a sample is performed using the matrix-assisted laser desorption/ionization mass spectrometer, and
the data acquisition step may acquire mass spectrum data of a plurality of fragment ions by performing a second measurement for detecting the fragment ions generated by dissociation of [M+H]⁺ or [M−H]⁻ of the nucleic acid under a modified analytical condition in which at least one analytical condition of the ion amount setting item, the mass-to-charge ratio range setting item, and the signal intensity setting item among the standard analytical conditions acquired in the analytical condition acquisition step is changed.

This makes it possible to detect fragment ions necessary for structural analysis with high sensitivity in a relatively wide mass region from a low mass region to a high mass region, and perform structural analysis of nucleic acids more reliably.

### Clause 7

In the nucleic acid analytical method according to clause 6,
the modified analytical condition may be a modified analytical condition in which the analytical condition of the ion amount setting item is changed so that the amount of ions generated in the ion source increases as compared with when the first measurement of the sample is performed under the standard analytical condition, a modified analytical condition in which the analytical condition of the mass-to-charge ratio range setting item is modified so that ions on a lower mass side than the mass-to-charge ratio of [M+H]⁺ or [M−H]⁻ of the nucleic acid are preferentially captured, or a modified analytical condition in which the analytical condition of the signal intensity setting item is modified so that the signal intensity of ions in the detection unit increases.

This makes it possible to detect fragment ions on a low mass-to-charge ratio side among fragment ions necessary for structural analysis with high sensitivity, and perform structural analysis of nucleic acids more reliably.

### Clause 8

In the nucleic acid analytical method according to clause 6 or 7,
the mass-to-charge ratio range setting item may be a time from when the ion source emits laser light to when a capturing voltage for capturing ions is applied to the ion capturing unit, and
the modified analytical condition may be a modified analytical condition in which the time is changed to a value shorter than the standard analytical condition.

This makes it possible to detect fragment ions on a low mass-to-charge ratio side among fragment ions necessary for structural analysis with high sensitivity, and perform structural analysis of nucleic acids more reliably.

### Clause 9

In the nucleic acid analytical method according to clause 8,
the modified analytical condition may be a modified analytical condition in which the time is set to a value shorter by 1 to 3 μs than the standard analytical condition.

This makes it possible to detect fragment ions on a low mass-to-charge ratio side among fragment ions necessary for structural analysis with higher sensitivity, and perform structural analysis of nucleic acids more reliably.

### Clause 10

In the nucleic acid analytical method according to any one of clauses 1 to 9,
the matrix-assisted laser desorption/ionization mass spectrometer may be of a digital ion trap type.

This makes it possible to detect [M+H]⁺ or [M−H]⁻ of the nucleic acid and fragment ions generated by dissociation of the ions with higher sensitivity.

### Clause 11

A matrix for matrix-assisted laser desorption/ionization mass spectrometry of a nucleic acid according to a mode of the present invention includes
3-hydroxypicolinic acid and 2,4-dihydroxyacetophenone, or includes 3-hydroxypicolinic acid and 2,4,6-trihydroxyacetophenone monohydrate.

This makes it possible to detect [M+H]⁺ or [M−H]⁻ of the nucleic acid and fragment ions generated by dissociation of the ions with high sensitivity.

### REFERENCE SIGNS LIST

1 . . . Ion Source
2 . . . Ion Trap
3 . . . Detection Unit
11 . . . Laser Irradiation Unit
12 . . . Sample Stage
21 . . . Ring Electrode
22, 23 . . . End Cap Electrode
31 . . . Conversion Dynode
32 . . . Detector (Secondary Electron Multiplier)

### SEQUENCE LIST

1. A nucleic acid analytical method, comprising performing mass spectrometry on a nucleic acid contained in a sample with a matrix-assisted laser desorption/ionization mass spectrometer using a mixed matrix containing 3-hydroxypicolinic acid and 2,4-dihydroxyacetophenone or a mixed matrix containing 3-hydroxypicolinic acid and 2,4,6-trihydroxyacetophenone monohydrate.

2. The nucleic acid analytical method according to claim 1, wherein when the mixed matrix is a mixed matrix containing 3-hydroxypicolinic acid and 2,4-dihydroxyacetophenone, a mixing ratio of 3-hydroxypicolinic acid and 2,4-dihydroxyacetophenone is 10:1 to 1:5.

3. The nucleic acid analytical method according to claim 1, wherein when the mixed matrix is a mixed matrix containing 3-hydroxypicolinic acid and 2,4,6-trihydroxyacetophenone monohydrate, a mixing ratio of 3-hydroxypicolinic acid and 2,4,6-trihydroxyacetophenone monohydrate is 1:1 to 1:3.

4. The nucleic acid analytical method according to claim 1, wherein the mixed matrix further contains ammonium citrate dibasic as a matrix additive.

5. The nucleic acid analytical method according to claim 1, comprising:
a data acquisition step of performing mass spectrometry on a sample containing the nucleic acid using the mixed matrix to acquire mass spectrum data of a plurality of fragment ions generated by dissociation of [M+H]⁺ or [M−H]⁻ of the nucleic acid; and
a data analysis step of extracting peaks of the plurality of fragment ions derived from the nucleic acid from the mass spectrum data and determining a structure of the nucleic acid based on mass information of the peaks.

6. The nucleic acid analytical method according to claim 5, wherein
the matrix-assisted laser desorption/ionization mass spectrometer is an ion trap type matrix-assisted laser desorption/ionization mass spectrometer including: an ion source performing a matrix-assisted laser desorption/ionization method; an ion capturing unit configured to separate and capture ions having a predetermined mass-to-charge ratio from ions generated by the ion source; and a detection unit configured to detect ions captured by the ion capturing unit,
the nucleic acid analytical method comprises an analytical condition acquisition step of acquiring standard analytical conditions of an ion amount setting item regarding an amount of ions generated by the ion source, a mass-to-charge ratio range setting item regarding a mass-to-charge ratio range of ions captured by the ion capturing unit, and a signal intensity setting item regarding signal intensity of ions in the detection unit when a first measurement for detecting [M+H]⁺ or [M−H]⁻ of the nucleic acid contained in a sample is performed using the matrix-assisted laser desorption/ionization mass spectrometer, and
the data acquisition step acquires mass spectrum data of a plurality of fragment ions by performing a second measurement for detecting the fragment ions generated by dissociation of [M+H]⁺ or [M−H]⁻ of the nucleic acid under a modified analytical condition in which at least one analytical condition of the ion amount setting item, the mass-to-charge ratio range setting item, and the signal intensity setting item among the standard analytical conditions acquired in the analytical condition acquisition step is changed.

7. The nucleic acid analytical method according to claim 6, wherein the modified analytical condition is a modified analytical condition in which the analytical condition of the ion amount setting item is modified so that the amount of ions generated in the ion source increases as compared with when the first measurement of the sample is performed under the standard analytical condition, a modified analytical condition in which the analytical condition of the mass-to-charge ratio range setting item is modified so that ions on a lower mass side than the mass-to-charge ratio of [M+H]⁺ or [M−H]⁻ of the nucleic acid are preferentially captured, or a modified analytical condition in which the analytical condition of the signal intensity setting item is modified so that the signal intensity of ions in the detection unit increases.

8. The nucleic acid analytical method according to claim 6, wherein the mass-to-charge ratio range setting item is a time from when the ion source emits laser light to when a capturing voltage for capturing ions is applied to the ion capturing unit, and
the modified analytical condition is a modified analytical condition in which the time is changed to a value shorter than the standard analytical condition.

9. The nucleic acid analytical method according to claim 8, wherein the modified analytical condition is a modified analytical condition in which the time is set to a value shorter by 1 to 3 μs than the standard analytical condition.

10. The nucleic acid analytical method according to claim 1, wherein the matrix-assisted laser desorption/ionization mass spectrometer is of a digital ion trap type.

11. A matrix for matrix-assisted laser desorption/ionization mass spectrometry of a nucleic acid, comprising 3-hydroxypicolinic acid and 2,4-dihydroxyacetophenone, or comprising 3-hydroxypicolinic acid and 2,4,6-trihydroxyacetophenone monohydrate.
